(19) United States
(12) Patent Application Publication
(10) Pub. No.: US 2026/0217794 A1
(43) Pub. Date: Jul. 30, 2026

(54) SYSTEM AND METHOD FOR PURIFICATION OF IMMUNOGLOBULINS FROM A BIOLOGICAL SAMPLE

(71) Applicant: KAMADA LTD., Rehovot (IL)

(72) Inventors: Yair PILPEL, Rehovot (IL); Roberto MEIDLER, Rehovot (IL)

(21) Appl. No.: 19/142,773

(22) PCT Filed: Dec. 24, 2023
(86) PCT No.: PCT/IL2023/051301

§ 371 Date: Jun. 24, 2025

Related U.S. Application Data
(60) Provisional application No. 63/436,719, filed on Jan. 3, 2023.

(51) Int. Cl. C07K 16/06; C07K 1/18; C07K 1/34; C07K 1/36

(52) U.S. Cl. CPC C07K 16/065; C07K 1/18; C07K 1/34; C07K 1/36; C07K 2317/14

(57) ABSTRACT

Provided herein are systems, devices, kits and methods for automatic purification and concentration of immunoglobulins from a biological sample. In particular, the system and methods disclosed herein facilitate the purification and concentration of immunoglobulins, such as IgG, from blood-derived plasma samples.

### TECHNICAL FIELD

The present disclosure relates generally to systems and methods for purification of immunoglobulins from the class IgG from a biological sample, such as blood derived plasma.

### BACKGROUND

Fractionated plasma products and in particular Intravenous Immunoglobulins (IVIG) are essential medications in accordance with the world health organization (WHO). Nevertheless, the availability of fractionated plasma products is imbalanced at a global level, and accordingly patients in low and medium income countries (LMIC) face severe difficulties to receive safe and efficient treatment, when available. In many LMIC countries, plasma-derived and recombinant products are not available in sufficient quantities, thereby exposing patients to crude blood or plasma fractions not subjected to virus inactivation treatment and susceptible to induce various immunological and bleeding side-effects. One of the major reasons is the lack of sufficient infrastructure and GMP facilities and insufficient funding for the import of such medications from developed (high income) countries. Likewise, particular plasma-derived treatments like hyper-immune IgG (or HIG). HIGs are generated from the blood plasma of donors that have high neutralizing antibody titers against specific viral diseases that are gained either through vaccination (e.g. Anti-Rabies [Anti-R]) or previous exposure or infection with a disease, or even immunization of animals and usage of their plasma. HIG is useful in many settings such as: pandemics (such as the Covid-19 outbreak), regional outbreaks of dangerous diseases (e.g. Ebola), arboviruses which tend to be locally transmitted (viral diseases transmitted by arthropods such as Yellow Fever, West Nile Virus, Zika), regional emergence of a novel strain of a known virus (Influenza, Covid), and the like.

Currently, the production of such medically relevant fractionated plasma products, and in particular immunoglobulins, are cumbersome, require a developed infrastructure of GMP factories and is costly and time consuming.

There is thus a need in the art for systems, devices and methods for purification and concentration of immunoglobulins from biological sample, which are able to operate at small scales, are versatile and capable to work outside of large scale GMP facilities (e.g. in a blood bank or field setting), are cost and time effective and which can be utilized on-demand at various locations.

### SUMMARY

Aspects of the disclosure, according to some embodiments thereof, relate generally to advantageous device, methods, kits and systems for separation of immunoglobulins from blood sample in a sterile, cost effective and time efficient manner, while being portable and having the capacity to operate at small scale, thereby allowing the implementation thereof in various settings, such as, blood banks, hospitals, health care facilities, field conditions, and the like.

According to some embodiments, there are thus provided herein systems, devices, kits and methods for a cost and time efficient, on-site, minimized separation, purification and concentration of immunoglobulins, such as Immunoglobulin G (IgG), from blood samples, in particular, blood plasma samples. In some embodiments, as detailed herein, the advantageous system includes a device (also referred to herein as apparatus or electromechanical assembly), and a kit which includes, inter alia, a set of corresponding vessels (for example, in the form of sterile bags), filters and/or separation columns, for the efficient purification of immunoglobulins, such as, IgG, from plasma samples. According to some embodiments, the system disclosed herein facilitates the passage of plasma samples/fractions via corresponding tubes, filters, buffers, separation columns (such as ion-exchange columns), in a sequential and coordinated manner, to allow the purification of the immunoglobulins from the blood samples, while maintaining sterility of the produced immunoglobulins, and while allowing concentration thereof to produce a concentrated, purified, preferably viral and bacterial free immunoglobulin extract (composition) that can be used for various medicinal, therapeutic or other purposes.

According to some embodiments, the system, device, kit and method can advantageously allow generating purified Immunoglobulin G (IgG) with no residual prothrombotic activity (activated prothrombin antecedent A.K.A. FXIa activity), and in some embodiments can allow generating extremely pure IgG with no residual detectable levels of other immunoglobulin classes such as IgA. In some embodiments, the system may be used in hospital blood bank or blood establishment based manufacturing from a relatively small number of donors (a “minipool”). As detailed herein, the system, device and kits are self contained and require no infrastructure except standard blood bank processing devices (e.g. sterile connection and disconnection devices). Advantageously, a single user (operator) can setup and operate the system in a time frame of minutes.

According to some embodiments, the system disclosed herein is particularly useful for treating viral diseases where the virus in question is of a rapidly evolving type such as Influenza or Covid, and the rate of pharmaceutical development is insufficient to cope with the rate of viral mutagenesis. The system can thus enable generation of a specific HIG concurrently within a local wave of infection and disease.

According to some embodiments, the systems, device, kits and methods disclosed herein, can, inter alia, be advantageously be used for: Generating hyper-immune IgG “on demand” from convalescent patient plasma for emerging epidemics or local outbreaks (e.g. Covid-19 and Influenza); Generating hyper-immune IgG for small indications that do not justify a full pharmaceutical GMP development; Generating IVIG and/or hyper-immune IVIG in low and medium income countries (LMIC) by utilizing their own blood banks and donor plasma; generating immunoglobulin compositions which are sterile, safe for use and which exhibit reduced adverse immunological side effects, such as, blood type incompatibility and HLA-related reactions such as life-threatening Transfusion Associated Lung Injury (TRALI) and others.

According to some embodiments, there is provided a device for automatic purification and concentration of immunoglobulins from a blood plasma sample, the device includes: one or more pumps; one or more valves; and a plurality of connecting elements configured to hold corresponding buffer vessels and/or bio-process vessels, wherein, when said plasma sample is introduced to the device, a sequential purification process is facilitated, whereby plasma sample fractions (i.e., fractions formed during the purification process, also referred to as “process intermediates”) are transferred between one or more of the bio-process vessels, such that immunoglobulins are purified, concentrated and collected at a collecting vessel, wherein the transferring of the fractions is facilitated by a coordinated operation of the one or more pumps and the one or more valves.

According to some embodiments, the device may be portable.

According to some embodiments, the device may further include a control unit configured to control operation of the one or more pumps and one or more valves. According to some embodiments, the control unit may be configured to control timing of operation, length of operation and/or sequence of operation.

According to some embodiments, the purified immunoglobulins include hyper immune IgG (HIG).

According to some embodiments, the purified immunoglobulin composition may include at least about 90% IgG.

According to some embodiments, the IgG composition may include IVIG (intravenous IgG), SCIG (subcutaneous IgG) and/or IMIG (intramuscular IgG).

According to some embodiments, the purified immunoglobulin composition does not exhibit residual prothrombotic activity and/or wherein the level of FXIa activity is less than about 10 mIU/ml.

According to some embodiments, the level of FXIIa (PKA) in the purified immunoglobulin fraction is less than about 35 IU/ml. According to some embodiments, the level of one or more of the following proteins in the collected immunoglobulin fraction is below a detection threshold of: plasminogen (less than about 0.01 gr/L), Alpha 1 antitrypsin (less than about 0.04 gr/L), albumin (less than about 0.0022 gr/L), IgM (less than about 0.05 gr/L) and IgA (less than about 0.06 gr/L).

According to some embodiments, the plasma sample may include a plasma pool of at least 4 plasma units. In some embodiments, the plasma is convalescent plasma. According to some embodiments, the plasma units may be obtained from different donors.

According to some embodiments, the device may be configured to allow purification of immunoglobulins in a time frame of about 6-48 hours.

According to some embodiments, one or more of the connecting elements may include weight transducers configured to determine the weight of a connected vessel.

According to some embodiments, the device may be operated automatically or semiautomatically.

According to some embodiments, the device may have a portable frame, allowing the movement or placement of the device at discrete locations.

According to some embodiments, there is provided a sterile kit for automatic purification and concentration of immunoglobulins from blood plasma sample, the kit includes: a plurality of reagent vessels, comprising one or more mixtures for use during the purification process; a plurality of bio-process vessels, configured to facilitate purification steps; one or more filtration units; and one or more separation columns; wherein the kit is configured to associate with a corresponding separation device, to allow sequential transfer of plasma sample fractions between the bio-process vessels, one or more filtration units and/or the one or more separation columns, to result in purified concentration immunoglobulin composition, collected at a collection vessel.

According to some embodiments, the kit may further include tubing elements configured to allow fluid connection between reagent vessels, bio-process vessels, filtration unit(s) and/or separation column(s) According to some embodiments, the reagent vessels may include mixtures such as, but not limited to: buffers, acetic acid, caprylic acid, citrate, glycine, TRIS, NaCl, water, amino acids, HEPES, MOPS, Carbonate, Oxalate, or any combination thereof, wherein said mixture are capable of being transferred to one or more corresponding bio-process vessels.

According to some embodiments, the bio-process vessels are configured to allow/accommodate incubation, mixing and/or reaction steps of the purification process. According to some embodiments, the vessels are made of such materials as, but not limited to: nylon, plastic, PVC, silicon, PP, PES, HDPE, BPC, or any combination thereof.

According to some embodiments, the vessels may be different in size, structure and/or composition.

According to some embodiments, the vessels comprise one or more connections or ports, allowing connecting between the vessels and/or between components of the separation device, using tubing elements.

According to some embodiments, the separation columns may include ion exchange columns. According to some embodiments, the ion exchange columns comprise a resin having a functional group selected from: Carboxymethyl cellulose (CM), Sulfonate(S), Sulfopropyl (SP), diethylaminoethyl (DEAE), quaternary aminoethyl (QAE), diethylaminopropyl (ANX) and/or quaternary ammonium (Q). In some embodiments, the resin may be selected from multimode ligand resins such as MEP (Mercaptoethylpyridine), or affinity ligand resins such as protein A, protein G, or protein L.

According to some embodiments, the kit may be disposable.

According to some embodiments, kit may be fabricated in a shape, size and/or form to fit the corresponding separation device.

According to some embodiments, the kit may further include a collection vessel, configured to collect/hold the purified immunoglobulins.

According to some embodiments, the immunoglobulin composition comprises IgG having a purity of over about 90%.

According to some embodiments, there is provided a system for purification of immunoglobulins from a blood plasma sample, the system includes the device and kit as disclosed herein.

According to some embodiments, there is provided a method for automatic purification and concentration of IgG composition from a blood plasma sample, the method includes providing a plasma sample in a vessel to the system disclosed herein; allowing fractions of the plasma sample to transfer sequentially between bioprocess vessels, one or more filtration units and/or one or more separation columns; and collecting the purified IgG composition, in a collection vessel.

According to some embodiments, method includes a step of associating the kit disclosed herein with the device disclosed herein, prior to providing the plasma sample.

According to some embodiments, allowing fractions of the plasma sample to transfer sequentially includes automatic transfer between one or more bioprocess vessels, one or more filtration units and/or one or more separation column, wherein said automatic transfer is controlled by a controller of the purification device.

According to some embodiments, the method further includes sealing a filled collection vessel, prior to removing the filled collection vessel.

According to some embodiments, allowing fractions of the plasma sample to transfer sequentially between bioprocess vessels, one or more filtration units and/or one or more separation columns includes one or more of the steps of: adding a reagent to a bioprocess vessels comprising a plasma fraction; mixing in a bioprocess vessels plasma fraction with a reagent, passing a mixed plasma fraction though a filtration unit; passing a wash buffer through a filtration unit, facilitating a chromatography reaction of a plasma fraction in a separation column; passing elution buffer through a separation column; filtering a plasma fraction to remove or inactivate pathogens; pasteurization of an immunoglobulin fraction. concentrating purified the immunoglobulin by passing through an ultrafiltration/diafiltration unit; exchanging the buffer to a formulation buffer by passing through the ultrafiltration/diafiltration unit; collecting concentrated immunoglobulins in a collection bag; adding a formulation buffer (wherein the formulation buffer may include, for example, stabilizer and/or any other suitable excipient to the concentrated immunoglobulins in a collection bag, or any combination thereof.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1A show a schematic perspective view of a device for purification and concentration of immunoglobulins, according to some embodiments;

FIG. 1B show a schematic front view of a device for purification and concentration of immunoglobulins, according to some embodiments;

FIG. 2 show a schematic perspective view of a kit for purification and concentration of immunoglobulins, according to some embodiments;

FIGS. 3A-C show schematic perspective views of a system for purification and concentration of immunoglobulins, according to some embodiments; FIG. 3A shows an assembled system; FIG. 3B shows system while biological sample vessel is being connected thereto; FIG. 3C shows the system and a schematic arrow illustrating the fluid flow direction in the system;

FIGS. 4A-B show schematic close-up views of a connecting element of a purification system, according to some embodiments;

FIG. 4C shows a schematic close-up view of a portion of a pump and associated tubing of a purification system, according to some embodiments;

FIG. 4D shows a schematic close-up view of a valve and associated tubing of a purification system, according to some embodiments;

FIG. 4E shows a schematic close-up view of ion exchange units of a purification system, according to some embodiments;

FIG. 4F shows a schematic close-up view of a collection vessel configured to hold and store the purified immunoglobulins; according to some embodiments;

FIG. 5 shows a block diagram of a method for purification of immunoglobulins, according to some embodiments;

FIG. 6 shows a pictogram of Western Blot analysis of purification of immunoglobulins from Plasma sample;

FIG. 7 shows MSD analysis of the final product after the purification process, revealing a highly purified IgG product; and

FIGS. 8A-E show pasteurization of purified IgG under different conditions. FIG. 8A shows bar graph of quantification of IgG monomers and dimers after pasteurization; FIG. 8B shows bar graph of quantification of aggregates; FIG. 8C shows Sample trace of product after DEAE at T₀; FIG. 8D shows Sample trace of product after DEAE after 5 hours at 50 C (in Tris); FIG. 8E shows Anti-Covid activity at 50 C for both Tris and Acetate buffer formulations.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application the expression “at least one of A and B”, (e.g. wherein A and B are elements, method steps, claim limitations, etc.) is equivalent to “only A, only B, or both A and B”. In particular, the expressions “at least one of A and B”, “at least one of A or B”, “one or more of A and B”, and “one or more of A or B” are interchangeable.

In the description and claims of the application, the words “include” and “have”, and forms thereof, are not limited to members in a list with which the words may be associated.

In figures depicting block diagrams/flowcharts, optional elements/steps may be written within a box delineated by a dashed line.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

As used herein, according to some embodiments, the term “kit” refers to a plurality of elements/units/components which may be used for a (common) specific purpose. i.e., for the separation/purification/concentration of immunoglobulins from a biological sample. As used herein, the terms “vessel”, “bag”, “chamber”, and “container” are interchangeable. The terms are directed to a container configured to accept fluids thereto, release fluids therefrom and/or hold/retain fluids within. In some embodiments, the vessel is sterile. In some embodiments, the vessel may be flexible, semi-rigid or rigid. In some embodiments, the vessel may be made of any type of biocompatible plastic. In some embodiments, the vessel may be made of any suitable material, including, for example, but not limited to: silicon, PES, Polypropylene (PP), plastic, nylon, PVC, BPC, HDPE, or any combinations thereof. In some embodiments, the vessel may be sterile or may be capable of being sterilized. In some embodiments, the vessel may have one or more ports, allowing receiving fluids thereto and/or releasing fluids therefrom.

As used herein, the term “reagent vessel” is directed to a vessel which is configured to hold and/or release a reagent composition/mixture/fluid which may be used in the purification process of immunoglobulins. In some embodiments, such reagent compositions may be selected from, but not limited to: buffers, solutions, precipitating agents, biological factors, aqueous solutions, water, or any combination thereof. In some embodiments, the reagent vessels are configured to hold and release the corresponding composition in a timely/coordinated manner to another reagent vessel or to one or more process vessels, in which a step of purification of one or more fractions of the biological sample may occur. In some embodiments, a reagent vessel does not include a fraction/portion of the biological sample.

As used herein, the term “bio-process vessel” is directed to a vessel which is configured to accept, release and/or hold at least a portion of the biological sample (for example, plasma fraction), optionally with a reagent composition. In some embodiments, the bio-process vessel is configured to allow/accommodate one or more of the purification steps of immunoglobulins. In some embodiments, a bio-process vessel may allow incubation of at least a portion of the biological sample with a corresponding reagent composition for a period of time.

As used herein, the term “collection vessel” is directed to a vessel which is configured to accept and hold purified, concentrated immunoglobulins, at the end of the purification process executed by the system. In some embodiments, the collection vessel may further allow releasing the purified immunoglobulins therefrom. In some embodiments, the collection vessel may be sealed after the purified immunoglobulins are transferred thereto.

According to some embodiments, the term “waste vessel” is directed to a vessel which is configured to accept, hold and optionally release fluids or other materials formed or collected during the purification process, which are not part of the purified immunoglobulin composition. Such materials may include, for example, but not limited to: buffers used for equilibration of chromatography resins, unbound fractions including proteins (that are not immunoglobulins), and the like.

In some embodiments, a plurality of vessels may be used in the kits and systems disclosed herein. The plurality of vessels may be similar, identical or different with respect to one or more of: size (volume), composition, shape and/or connectors. Each possibility is a separate embodiment.

According to some embodiments, the term “separation column” is directed to a separation unit configured to allow separation of molecules based on one or more physiological, functional and/or chemical properties, such as, but not limited to: electrical charge, size, biological activity, affinity, and the like, or any combinations thereof. In some embodiments, the separation column may be a chromatographic column, ion exchange columns, such as, anion exchange column and cation exchange column, and the like. In some embodiments, the separation column may include any suitable resin, such as, for example, Sepharose, agarose, ceramic, or other type of resin matrix, that may include functional groups, such as, for example, but not limited to: Carboxymethyl cellulose (CM), Sulfonate(S), Sulfopropyl (SP), diethylaminoethyl (DEAE), quaternary aminoethyl (QAE), diethylaminopropyl (ANX), quaternary ammonium (Q), and the like, or any combinations thereof. In some embodiments, additionally, or alternatively, other resins may function using mixed mode such as MEP (Mercaptoethylpyridine), or by affinity to immunoglobulins such as protein A or protein G, or modifications thereof (e.g. Protein L).

According to some embodiments, the kit and/or system disclosed herein may include a plurality of separation columns that may be used at various stages/steps of the purification and concentration of the immunoglobulins. In some embodiments, the plurality of separation columns may be similar, identical, or different with respect to one or more of: size (volume), capacity, composition, type, form, shape, orientation and/or connectors. Each possibility is a separate embodiment.

According to some embodiments, the term“ filtration unit” and “filtration element”, may interchangeably be used. The terms are directed to physical elements which are used to filter precipitates, aggregates, molecules, microorganisms, impurities, and the like, during the purification and purification of the immunoglobulins. In some embodiments, the filter unit may be any type of filter having a size, shape, form, composition, capacity, cut-off value and/or pore size suitable for its use during the purification process. In some embodiments, the filtration unit may be made of rigid, semi-rigid, soft, flexible, and/or hard materials. In some exemplary embodiments, the pore size may be in the range of from about 0.2 micron to about 75 micron, depending on the stage in the process in which the filtration is facilitated.

According to some embodiments, the kit and/or system disclosed herein may include a plurality of filtration units that may be used at various stages/steps of the purification and concentration of the immunoglobulins. In some embodiments, the plurality of filtration may be similar, identical or different with respect to one or more of: size, capacity, composition, type, form, shape, orientation, connectors and/or pore size. Each possibility is a separate embodiment.

According to some embodiments, the term “tubing elements” is directed to physical elements which are used to fluidly connect various components of the kit and system. In some embodiments, the tubing elements may include pipes, tubes, conduits that can connect to components of the kit or system to thereby form fluid connection between connecting elements, to facilitate fluid transfer. The tubing elements may be flexible, soft, hard, semi rigid, rigid. The tubing element may be made of any suitable polymeric or non-polymeric material, including, for example, silicon, PC, BPC, HDPE, PVC, PES, PP, plastic, and the like. In some embodiments, the tubing elements may include distinct elements connecting between adjacent components of the kit. In some embodiments, at least some of the tubing elements, or at least a portion of the tubing element, may interact/contact pump(s) and/or valves of the purification system. In some embodiments, the kit may include a plurality of tubing elements, that may be similar, identical or different with respect of one or more of: size (internal and/or external diameter), capacity, composition, type, form, shape, orientation, connectors and the like. Each possibility is a separate embodiment.

According to some embodiments, the term “biological sample ”is directed to a fluid sample obtained from a subject, wherein the biological sample includes immunoglobulins there within. In some embodiments, the biological sample is a blood sample or fractions thereof. In some embodiments, the biological sample is blood plasma sample. In some embodiments, the subject is a mammal. In some embodiments, the subject is human. In some embodiments, the subject is an animal, such as, a horse, a goat, a sheep, a pig, rabbit, mice, and the like. In some embodiments, the subject may have been afflicted with or immunized to a disease, such as, for example, but not limited to: viral disease (such as, for example, Covid-19, Influenza, CMV, RSV, WNV, Zika, Dengue, and the like). In some embodiments, animals may be vaccinated with a specific toxin such as a venom from a snake, spider, or scorpion, or other venomous animal so as to enable the extraction of an anti-venom hyper-immune globulin.

As used herein, the terms “plasma” and “blood plasma” may interchangeably be used. The terms are directed to plasma fraction of blood, i.e., blood fluid which does not include blood cells. In some embodiments, the plasma may be obtained from a donor (a single donor or a plurality of donors). In some embodiments, the donor(s) are convalescent donors (i.e., donors recovering from a disease). In some embodiments, the plasma comprises apheresis plasma (“source plasma”). In some embodiments, the plasma is a recovered plasma (i.e. plasma separated from a whole blood sample). In some embodiments, the plasma is fresh plasma. In some embodiments, the plasma is frozen and thawed prior to use. In some embodiments, the plasma may be sourced locally. In some embodiments, the plasma may be obtained from plasma collection centers. In some embodiments, the plasma may be pooled (i.e., more than one plasma unit may be pooled together) prior to being processed by the system. In some embodiments, pooling plasma samples may be smart pooling, which takes into consideration mismatch-hemolytic reactions (i.e. may eliminate Transfusion-associated adverse events (such as transfusion associated lung injury (TRALI)) and hemolytic reactions due to blood group ABO blood groups incompatibility).

According to some embodiments, there is provided a device for separation, purification and/or concentration of immunoglobulins from a biological sample, such as, plasma sample. As detailed herein, the device may be in the form of an assembly which includes a stand/frame which holds various pumps, valves and connecting elements, all configured to facilitate the process of purification, when connected/associated with a corresponding kit, which includes the additional elements required for the purification process (i.e., various vessels, separation column(s), filtration units and corresponding tubing).

According to some embodiments, the device may advantageously be portable (i.e., cable of being moved between locations). In some embodiments, the device (in particular, the frame thereof) may include transportation means, such as, wheels, allowing movement thereof between different locations. According to some embodiments, the device may advantageously have a relatively small form factor, allowing its use in locations with limited space.

According to some embodiments, the device may further include a control unit, that may include a control circuitry configured to control functions/operations of components during the operation of the device. In particular, a control circuitry may include one or more processors, one or more (transient and non-transient) memory components, internal clock, and the like. In some embodiments, the control unit may control one or more operating parameters of components/units of the device, including, for example, but not limited to: timing of operation of various units, length of operation of various units, mode of operation of various units, order of operation, and the like. In some embodiments, the control unit may operate fully automatically, semi automatically, or be controlled manually.

According to some embodiments, the device may further include a user interface, allowing a user to determine operating parameters, input data and/or receive various data from the device, before, during and/or after operation. In some embodiments, the device may further include a power source, a communication unit (wired or wireless), and the like.

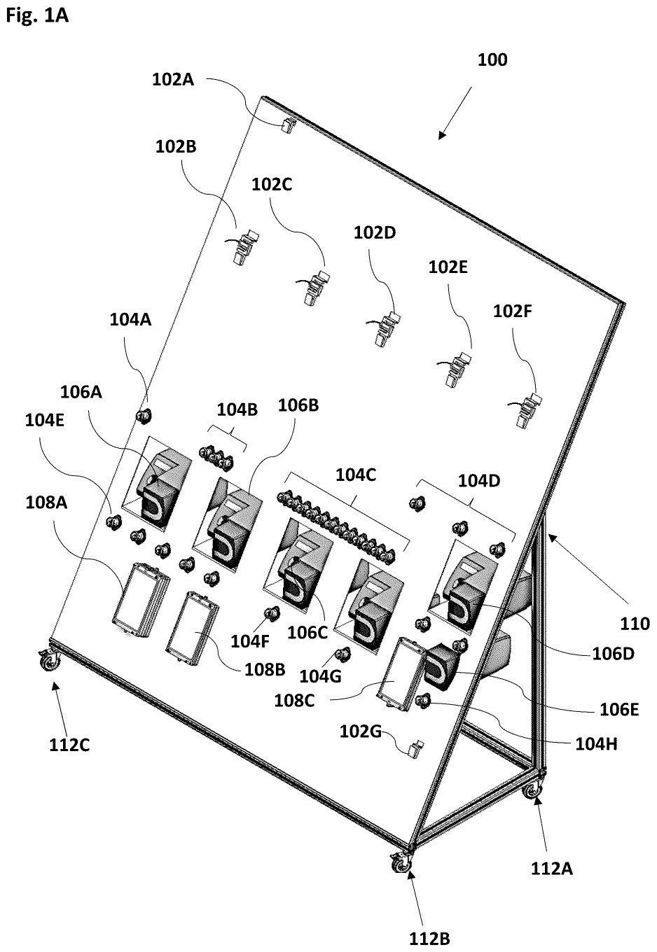

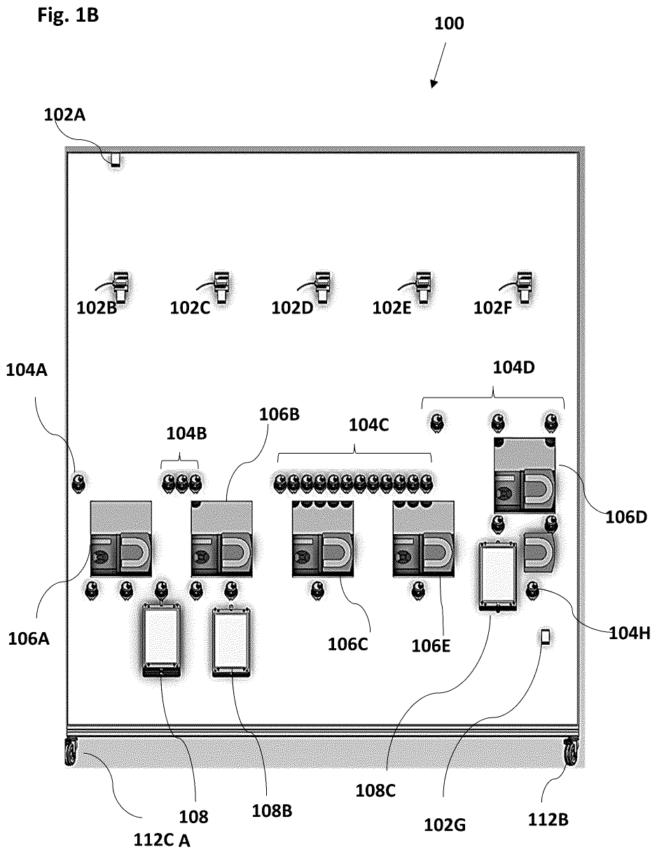

Reference is now made to FIGS. 1A-B, which show schematic views of a device for separation of immunoglobulins, according to some embodiments. Shown in FIG. 1A is a perspective view of device 100, and FIG. 1B shows a front view of the device. As shown, device 100 includes a frame 110, which may be horizontally angled, and may include transportation elements, shown as exemplary wheels 112A-C. Further shown are exemplary connecting elements 102A-G, which are configured to attach/associate/hold corresponding vessels (for example, bio-process vessels (e.g., connectors 102B-F), collecting vessel (e.g., connector 102G), biological sample vessel (e.g., connector 102A)) of a corresponding kit. In some embodiments, the connecting elements may be in the form of a clip, a hook, a screw, and the like. In some embodiments, the connecting elements may further function as a scale, capable of determining weight or volume of a connected vessel. In some embodiments, in such instances, the determined weight or volume may be used for determining/controlling one or more operating conditions of the device. Further shown are valves 104A-H (some of the valves shown in the figures are not marked). The valves may include any type of suitable valve, such as, for example, pinch valve, that may associate with tubing elements (once the kit is assembled to the device) and are configured to control fluid flow (for example, amount, rate, close/open) between components of the kit. Further shown are pumps 106A-E, that are capable of controlling flow of various mixtures (containing or not containing plasma fractions) between various components/units of the kit. As detailed herein, the pumps may include any type of pumps, such as, for example, peristaltic pumps. In some embodiments, the operation of the pumps may be controlled by a control unit (not shown), and the rate, speed, volume of operation may be controlled automatically, semi automatically or manually, based, inter alia, on the weight or volume of at least some of the connected vessels. Further shown are housing 108A-C, which are configured to accommodate corresponding filters of the kit, as detailed below.

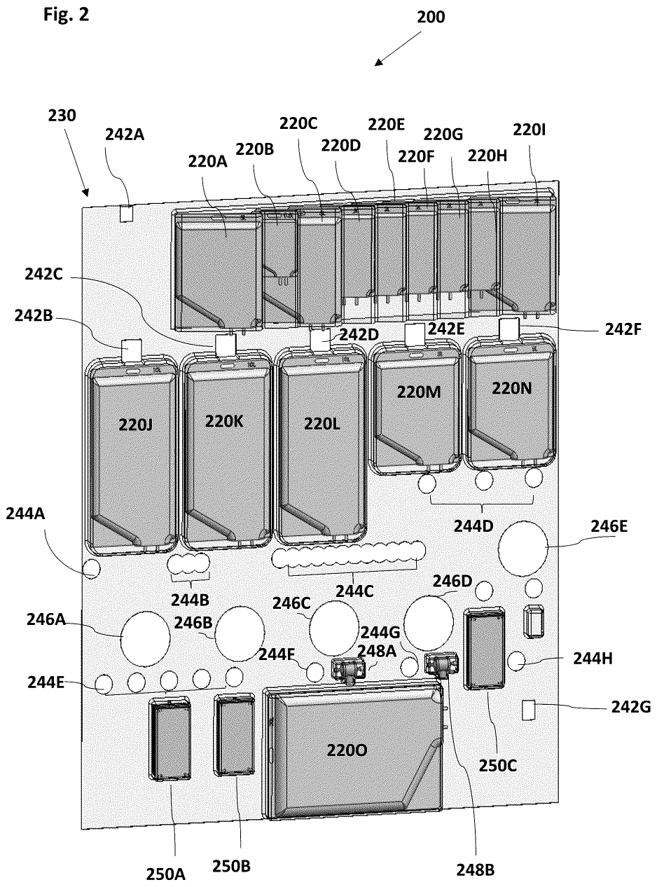

Reference is now made to FIG. 2, which shows an exemplary kit for purification of immunoglobulins, according to some embodiments. A shown in FIG. 2, kit 200, includes frame 230 that may be rigid, flexible or semi flexible. The frame may be used to connect/associate various other components of the kit. The kit may include one or more openings/apertures, to fit corresponding elements of the device (shown in FIGS. 1A-B). In FIG. 2, shown are exemplary openings 242A-G, which correspond to connecting elements 102A-G of the device (shown in FIG. 1A), openings 244A-H, which correspond to valves 104A-H (shown in FIG. 1A), openings 246A-E, which correspond to pumps 106A-E (shown in FIG. 1A). Kit 200 further includes a plurality of vessels (shown as vessels 220A-O). The vessels may be glued, adhered, associated, placed, attached to the body frame 230 of the kit. The vessels may be reagent vessels (for example, vessels 220A-I), bio-process vessels (for example, vessels, 220J-N), waste vessel (2200), and the like. Omitted from FIG. 2 is collection vessel, which is configured to hold/accept/receive the purified immunoglobulins. Further shown in FIG. 2, are separation columns 248A-B, and filtration units 250A-C. The various components of the kit (e.g., vessels, filtration units, columns, etc.) may be interconnected by tubing elements (not shown), allowing a fluid connection between various components.

According to some embodiments, the kit may be prefabricated or may be fabricated by a user. In some embodiments, the kit may be disposable. In some embodiments, the kit may be packed in the form of a blister. In some embodiments, the size, shape, form, dimension, composition of the kit may be adjusted to fit the corresponding device and/or the biological sample.

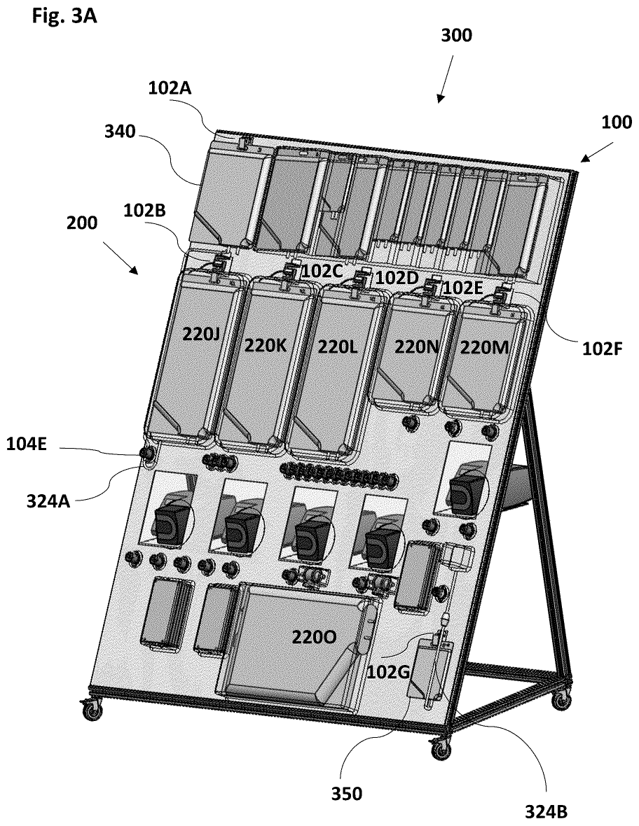

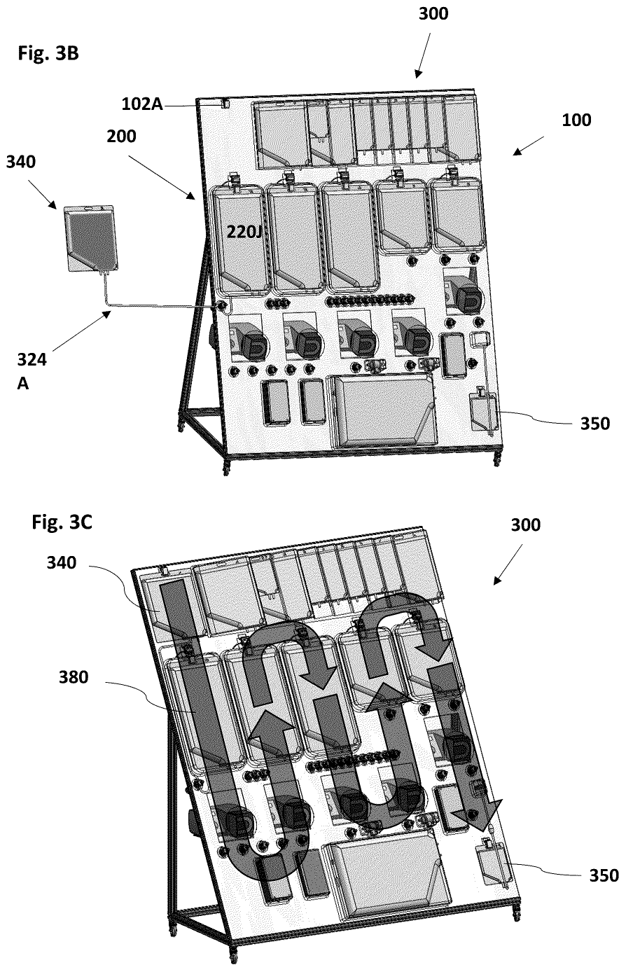

Reference is now made to FIGS. 3A-C, which show schematic illustrations of a system for purification of immunoglobulins, according to some embodiments. As shown in FIG. 3A, system 300, is comprised of device 100 (shown in FIGS. 1A-B), which is associated with kit 200 (shown in FIG. 2). As can be seen in FIG. 3, kit 200 is associated with device 100, such that the various elements of the kit fit one the device. For example, bioprocess vessels 220J-N are connected to the device via corresponding connecting elements 102B-F of the device. Further shown is collecting vessel 350, being connected to the device via connecting element 102G. Further shown is sample vessel 340, being connected to the device via connecting element 102A. Also shown is exemplary tubing element 324A, fitted through valve 104E, wherein tubing element 324A is configured to fluidly connect sample vessel 340 to bioprocess vessel 220J. Also shown is exemplary tubing element 324B, configured to allow passage of fluids to collecting vessel 350.

Reference is made to FIG. 3B, which shows sample vessel 340, being fluidly connected to bioprocess vessel 220J via tubing element 324A, prior to attaching (hanging, fixing) the sample vessel 340 to device 100, via connecting element 102. As shown in FIG. 3B, tubing element 324A is associated with valve 104E, which is configured to control fluid flow between the sample vessel and the bioprocess vessel 220J.

Reference is made to FIG. 3C, which schematically illustrates the directional and sequential purification process, whereby sample fractions are conveyed/transferred/moved from sample vessel 340, to a first bioprocess vessels, and thereafter further conveyed/transferred/moved therefrom to bioprocess vessels (which are further supplied with one or more reagent compositions from reagent vessels), filtering units and/or separation columns, to result in a purified and concentrated immunoglobulin composition which is collected in collection vessel 350. The process direction is marked by wide arrow 380. In some embodiments, the movement of fluids between the vessels, filtering units and/or columns may be facilitated or controlled, at least partially, by the valves and/or pumps of the device. In some embodiments, the movement of fluids between various components of the system may be according to gravitational forces. In some embodiments, the amount (volume) of fluids conveyed between the components of the system, rate of transfer, rate of flow, length of time the fluid is within or is in contact with a component of the system, direction of flow, and the like, may be controlled by the controller of the system (i.e., the controller of the device), and may be determined, at least partially based on the amount, volume, weight and/or type of the biological sample (for example, blood plasma), introduced to the system.

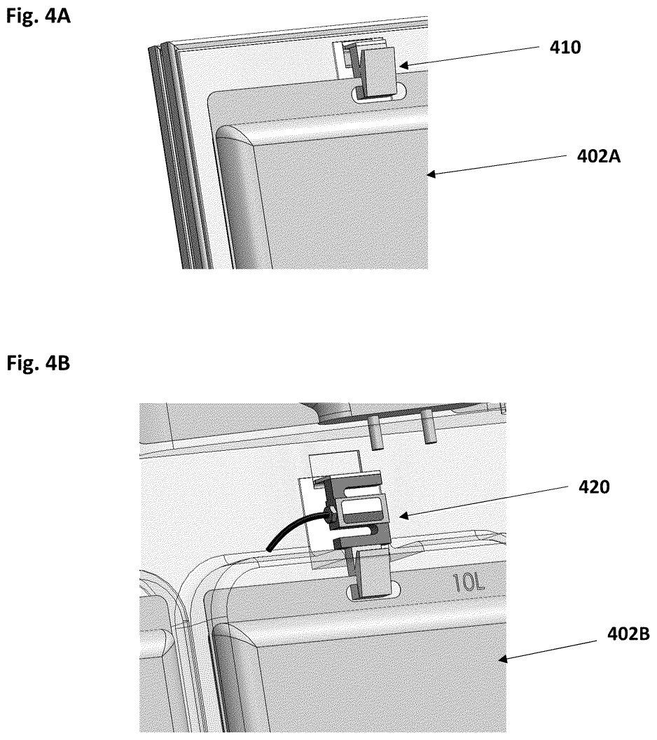

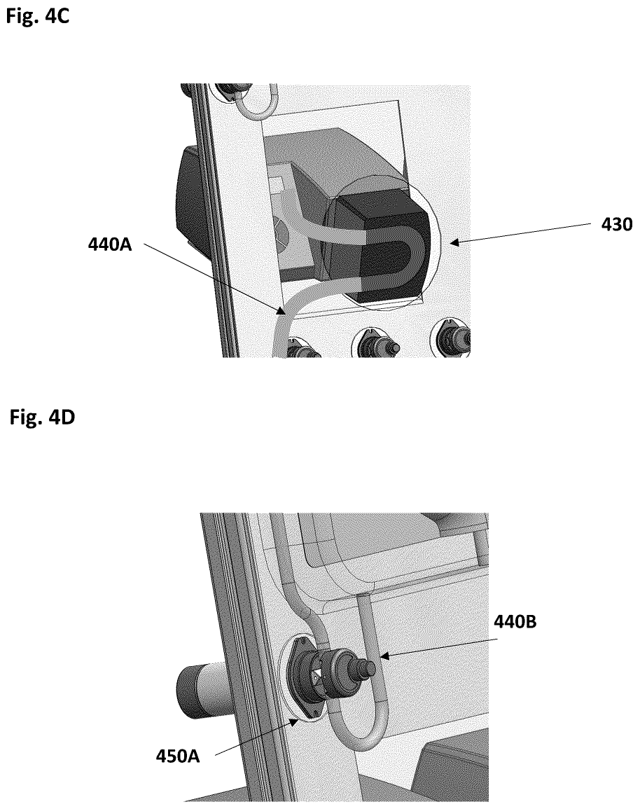

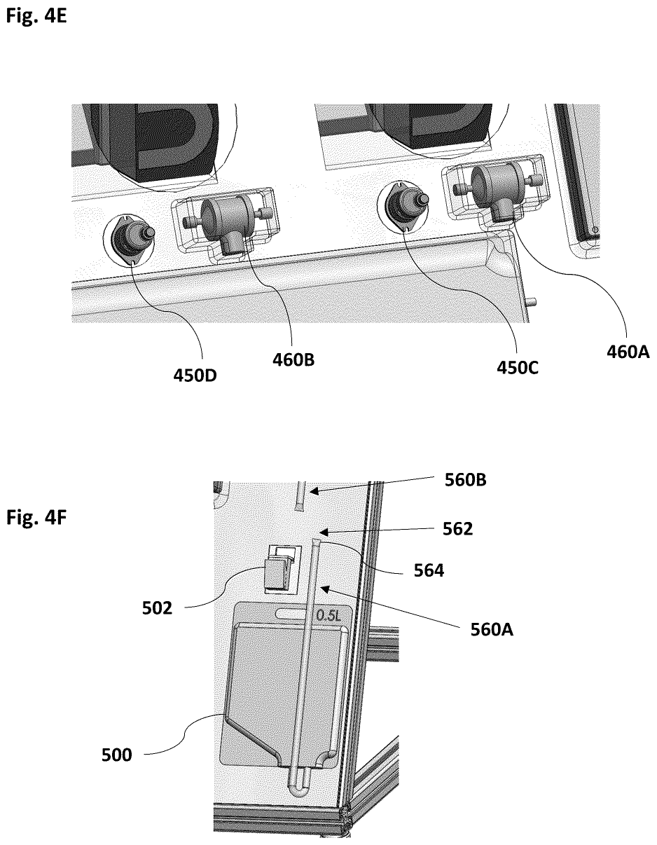

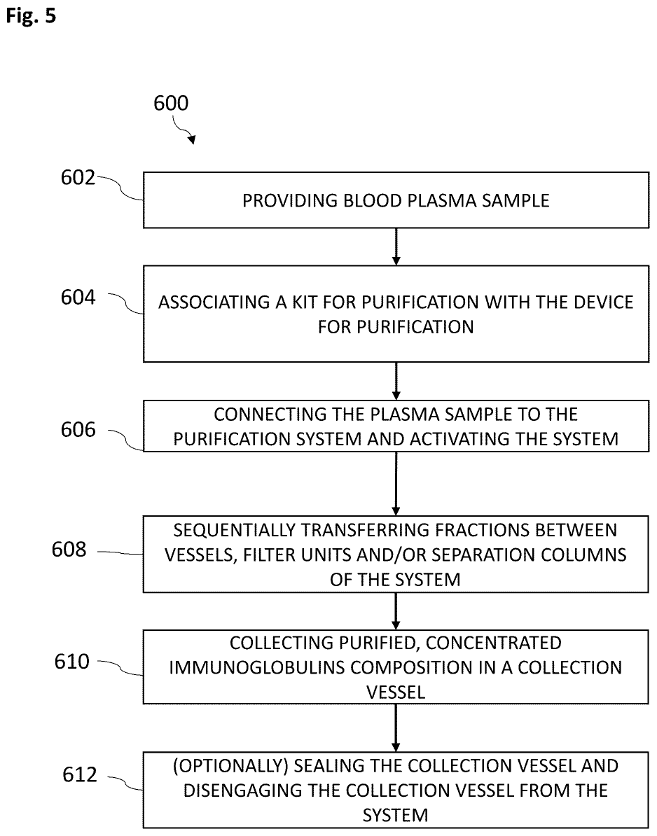

Reference is now made to FIGS. 4A-B which show schematic close-up views of a connecting element of a purification system, according to some embodiments. Shown in FIG. 4A is connecting element 410, in the form of a clip/holding pin, which is capable of attaching a corresponding vessel 402A, at a holding region/opening in the vessel. Shown in FIG. 4B is exemplary connecting clement 420, in the form of a clip/holding pin, which is further capable of determining weight and/or volume of the vessel associated therewith. In such instances, the connecting element may be a weight/scale element, capable of sensing the weight or volume of the connected vessel. The weight and/or volume information may be used for determining one or more operating parameters of the purification system or purification process, such as, for example, amount/volume of buffer or other reagents to be used at various stages, incubation time (length), and the like.

Reference is made to FIG. 4C, which shows a schematic close-up view of a portion of a pump and associated tubing element of a purification system, according to some embodiments. As shown in FIG. 4C, tubing element 440A is associated with pump 430, such that the pump can control/affect fluid flow within the tubing element, and hence, fluid flow in the system, between various components. In some embodiments, the pump may be used to convey fluids between vessels, filtration units and/or separation columns. In some embodiments, the pump may be used to facilitate mixing within a bioprocess vessel, for example, by recirculating fluid within the vessel (for example, by removing and re-adding the fluid to the vessel.

Reference is made to FIG. 4D which shows a schematic close-up view of a valve and associated tubing of a purification system, according to some embodiments. As shown in FIG. 4D, tubing element 440B is associated with valve 450A, which can control/affect fluid flow within the tubing element and fluid flow in the system, between various components. As shown in FIG. 4D, at least a portion of tubing element 440B is passed via valve 450A, thereby allowing the valve to control flow parameters via the tubing element, including, for example, rate of flow, amount of flow, time length of flow, timing of flow, and the like.

Reference is made to FIG. 4E which shows a schematic close-up view of separation columns of a purification system, according to some embodiment. As shown in FIG. 4E, separation columns 460A and 460B, each include an entrance port and an exit port and a column body, which harbors the separation media, such as, a resin that may be modified with various functional groups, depending on the type of separation (for example, anion or cation exchange). The separation columns of the system may be of different or similar size, shape, form, type and/or orientation. Further shown in FIG. 4E are valves 450D and 450C, which can control fluid flow to/from the separation columns.

Reference is made to FIG. 4F which shows a schematic close-up view of a collection vessel configured to hold and store the purified immunoglobulins; according to some embodiments. Shown in FIG. 4F is collection vessel 500, being disengaged from corresponding connecting element 502. Further shown are tubing elements portions 560B and 560 A, that fluidly connected vessel 500 to the purification system. As shown in FIG. 4F, once the purification process has been completed and the immunoglobulins have been collected in the collection vessel, the tubing elements may be disengaged/cut/disconnected, for example, at region 562, to allow releasing of collection vessel 500 from the system. In some embodiments, the disengagement may be performed by mechanical disengagement/cutting/disrupting the tubing element. In some embodiments, the mechanical disengagement may be facilitated by welding/heating the tubing element at a disengagement region (for example, region 562), using a heating unit of the system or a radiofrequency tube welder (such as is use in blood banks). In such a setting, end 564 of tubing 560A is sealed (due to the heating/welding thereof). By sealing distal end 564, the integrity and sterility of the collection vessel is maintained, allowing its disconnecting from the system and transfer for further use.

According to some embodiments, the device, kit and system disclosed herein are used for the purification of immunoglobulins from blood plasma, in a sequential, coordinated process, whereby plasma fractions/purification intermediates are conveyed in or between bio-process vessels (which are further added with suitable reagent mixtures from the reagent vessels), filtration units and separation columns, to result in purified, concentrated immunoglobulin compositions. According to some embodiments, the purification process may include one or more of the following steps disclosed herein.

According to some embodiments, the purification process executed by the system disclosed herein may include one or more of the following steps:
Connecting the biological sample vessel harboring the biological sample (for example, blood plasma) to the system. Optionally, the weight/amount/volume of the biological sample may be determined.
Adding/transferring a suitable volume/weight of a buffer from a first reagent buffer to a first bio-process vessel, into which the biological sample (e.g. plasma) is transferred from the biological sample vessel. The fluid flow of the sample and buffer are facilitated via the connecting tubing elements. In some embodiments, the ratio between the buffer and the plasma may be between about 0.5-2 buffer: plasma (for example, 1:1). In some embodiments, the buffer may include acetic acid. The transfer of the plasma to the bioprocess vessel may be facilitated by a corresponding pump (e.g. peristaltic pump), or by gravity flow. The addition/transfer of the equivalent weight or volume of the buffer is added to the same bio-process vessel as the plasma, wherein the addition is controlled by the valves (e.g., pinch valves) assembled/associated with the tubing elements.
Adding/transferring reagent mixture comprising a precipitating reagent (such as, for example, caprylic acid) at a suitable volume (for example, 4-6% volume of the total volume, or, for example, 8-12% of the initial plasma volume).
Mixing within the bioprocess vessel for a period of time (such as, for example, in the range of 10-120 minutes). In some embodiments, the mixing may be facilitated by a pump of the system, which can circulate the fraction via dedicated tubing elements from and back into the bioprocess vessel. In some embodiments, the mixing rate (i.e., flow rate of the pump may be in the range of about 50 ml/minute to 1000 ml/minute (for example, in the range of about 200 -500 ml/minute).
Filtration, whereby the mixed fraction is transferred via the tubing elements to a first (crude) filtering unit. The transferring is facilitated by the operation of a pump. In some embodiments, the filtering units have an area of approximately 1 cm² per 1 ml of input plasma. In some exemplary embodiments, the filter area may be in the range of about 1500-3500 cm² (for example, 2500 cm²), for an estimated volume of about 10-12 units of plasma, which typically ranges between 2000-3200 ml. In some embodiments, the filtration rate (i.e., the flow rate of the pump) may be in the range of about 50-500 ml/minute (for example, 100-200 ml/minute). In some embodiments, the filter pore size may be in the range of about 5 -75 micron.
Washing the filter with a suitable volume of a wash buffer (conveyed from a corresponding reagent vessel). In some embodiments, the washing step is performed by adding (for example, by pumping) a volume of washing buffer that is equivalent to the measured volume of the starting plasma. In some embodiments, the flow rates may be similar to those used to filter the plasma-mixture through the filter in the previous step. In some embodiments, the volume can be +/−10% lower or higher than the volume of the starting plasma. In some embodiments, the pH of the buffer may be adjusted or predetermined. In some embodiments, the buffer may have a pH in the range of 4.2-4.8. In some embodiments, the conductivity of the wash buffer may be between 1-3 milliSiemens/cm². In some embodiments, the wash buffer may include acetic acid.

Additionally, and alternatively, a second filter unit having a smaller area may be implemented at this stage to filter away residual aggregates or precipitates and increase purity.
chromatography purification via a separation column. At this stage, the fraction of the previous stage is conveyed to/via a cation exchange column. The cation exchange column may include any type of suitable resin and functional chemical groups, such as, for example, Carboxymethyl (CM), Sulfonate(S), or Sulfopropyl (SP). In some embodiments, the pH may be in the range of about 4.2-4.8. In some embodiments, the conductivity may be in the range of about 3-7 ms/cm². In some embodiments, the separation step using the column may be performed in several cycles, in order to enable utilization of a smaller amount of resin. The total effective resin volume is cumulative. I.e.—a 50 ml column used twice is equivalent to a single use of a 100 ml column. In these cases, the column is regenerated by methods known in the art such as a high salt or high pH solution, followed by a wash and equilibration step. As low as 1/10th of the volume may be loaded onto a column housing 1/10th of the nominal required resin volume, respectively. The column is regenerated after each cycle with a suitable buffer (for example, 0.5-2M NaCl) followed by equilibration with an equilibration buffer (such as, for example, acetic acid at pH 4.2-4.8), to allow the next cycle. In some embodiments, the volume of the columns may be between 30 and 600 ml (for example, between 50-300 ml, or between 50-60 ml). Optionally, more than one column may be used in parallel, and the fractions/purification intermediates may be divided accordingly.

The solution washed from the column may be discarded and transferred into the waste vessel, as the immunoglobulins are bound to the cation exchange resin of the separation column.
Elution step—the next step includes removal/elution of the antibodies from the cation exchange column and collection thereof into a separate bio-process vessel. The elution may be performed with a suitable elution buffer (for example Tris at pH in the range of 8.4-8.6, and NaCl).

The entire chromatography purification sequence may be repeated until all of the filtered precipitate and wash solution has been processed through the column.
Dilution step—The eluate collected in the bio-process vessel may be diluted for further processing. A dilution step may be required for the reduction of the conductivity of the solution to acceptable levels for the next processing step, i.e. reducing to between about 3-7 mSiemens/cm². In some embodiments, the eluate may be diluted by the addition of an equivalent amount of reagent mixture (such as, for example, water) into the bio-process vessel. In some embodiments, the ratio of the reagent mixture (e.g. water) to the eluate may be in the range of about 0.6:1 to 2.5:1 (for example, between 1:0.9 and 1:1.1).
Optionally—chromatography purification via a separation column, to remove unwanted immunoglobulins. Performance of an anion exchange flow-through purification step. This step can reduce the levels of protein aggregates to below 1%. Moreover, it can potentially remove any residual non-IgG protein, including the potentially anaphylactic IgA antibody class, in case IgG are the purified immunoglobulins. In some embodiments, the anion exchange column may include a with one of more of the functional groups: DEAE (diethylaminoethyl), QAE (quaternary aminoethyl), ANX (diethylaminopropyl), and Q (quaternary ammonium). In some embodiments, the amount of anion exchange resin in the column may be, for example, between 100-400 ml. In some embodiments, the chromatography step may be performed in several cycles, in order to enable the utilization of a smaller amount of resin. In such the column may be regenerated after each cycle e.g. by use of a high salt solution or other methods known in the art (e.g. low pH solution) followed by a wash and equilibration step. When using such anion exchange column, the diluted solution from the previous step (i.e., cation exchange column eluate diluted with water as detailed above) is loaded onto the anion exchange column and the material passing through the column is collected into the next bioprocess vessel. In this mode (i.e., flow-through), the unwanted impurities are retained on the anion exchange column, whereas the purified immunoglobulins solution passes through the column and is collected.

The final product after this step may include a highly purified (>99.9% monomer+dimer) IgG immunoglobulins, with no detectable impurities such as IgA (<0.06 mg/L).
Optionally—a second pathogen inactivation (sterilization) step may be carried out. As a commonly requested regulatory requirement is to maintain two orthogonal (of a different physicochemical basis) viral inactivation methods for PDMPs, a second inactivation step may be used. Thus, in addition to a precipitation step (for example, with caprylic acid), for increased safety and optionally also regulatory compliance in some cases, it is possible to integrate a pasteurization step for viral inactivation. To this aim, various stabilizers such as sorbitol or other suitable sugars (for example, at 15-40% w/w) may be added to the fraction, to protect the proteins therewithin. In some embodiments, the stabilizer may include a pharmaceutical grade sugar moiety such as sorbitol, sucrose, maltose, mannitol, or trehalose, and the like. In some embodiments, the stabilizer may be added to the bio-process vessel, at this stage (i.e., after the purification steps of precipitation (for example, by caprylic acid) and filtration, cation exchange chromatography, and the optional anion exchange chromatography. In some embodiments, the stabilizer is added from a concentrated stock solution stored/held in a corresponding reagent vessel in the kit. In some embodiments, the stock solution may be between 40-60% w/w dissolved in suitable solvent, such as, water. In some embodiments, the stabilizer solution may be added to the fraction a final concentration of between about 10-40% w/w (for example, about 15%-30%, for example, about 20-25%). In some embodiments, the resulting solution may be heated to a nominal temperature (for example, 50-65° C.), for example by a corresponding heating unit (such as heating element or heating pump) of the device and system. In some embodiments, the heating unit may include a plasma warmer unit, including a sterile-connected cassette that is heated via an electrical control. In some embodiments, the heating unit may include a water bath into which the corresponding bioprocess vessel may be moved while still interconnected with the kit so as not to breach sterility. In some embodiments, the heating unit may include an external heating device capable of transmitting heat in a controlled fashion to the corresponding tubing elements.

According to some embodiments, the sterilization solution may be maintained at a desired temperature for a desired period of time, such as, for example, 4-12 hours (for example, 5-10 hours), in order to enhance viral inactivation.

According to some embodiments, a final step in the purification process by the system and methods of the present disclosure may include a step of formulation and concentration of the purified immunoglobulins. In some embodiments, such as step may include removal of any residual reagents from the process (including, for example, buffers, solution, stabilizers, precipitation agents, etc.) and optionally replacement with a suitable formulation buffer, to form a final composition (which includes the purified immunoglobulins and optionally one of more solutions, excipients, etc.).

According to some embodiments, the formulation buffer may be selected in accordance with one or more of: the type of purified immunoglobulins, the amount/concentration of the purified immunoglobulins, the intended use (purpose) of the immunoglobulins, and the like.

In some exemplary embodiments, the formulation buffer is suitable/optimal for the purpose of IVIG. In some exemplary embodiments, the formulation buffer is suitable/optimal for the purpose of SCIG. In some exemplary embodiments, the formulation buffer is suitable/optimal for the purpose of IMIG.

In such embodiments, the formulation buffer may include any of a buffers and excipients that are generally regarded as safe for the purpose of parenteral biologics formulation, including, for example, but not limited to: sodium phosphate (for example, in the range of about 10-50 mM), sodium acetate, glycine, proline, lysine, arginine, histidine, glutamate, succinate, citrate, lactic acid, adipic acid, MES, aspartic acid, TRIS, or any buffer used in the formulation of immunoglobulin formulations. Additional excipients may be added to stabilize the product, reduce its viscosity, act as surfactant, or other purposes such as polysorbate 20, PEG e.g. PEG 3350, poloxamer 188, or other materials such as metal chelators e.g. EDTA or DTPA, or antioxidants such as methionine.

In some embodiments, the pH of the buffer may be in the range of about 4.0 to about 8.0, or any subranges thereof.

In some embodiments, the pH of the buffer may be in the range of about 4.8 to about 6.3.

In some embodiments, the pH of the buffer may be in the range of about 5.0 to about 6.0.

In some embodiments, the pH of the buffer may be in the range of about 5.2 to about 5.7.

In some embodiments, the osmolarity of the buffer may be in the range of about 250 to about 400 mOsmolar, or any subranges thereof. In some embodiments, the osmolarity of the buffer may be in the range of about 285 to about 335 mOsmolar. In some embodiments, the osmolarity of the buffer may be in the range of about 290 to about 315 mOsmolar.

According to some embodiments, the step of concentration may further include utilizing an ultrafiltration/diafiltration step (concentration and buffer exchange), preferably using a pre-sterilized membrane filter, having a suitable molecular weight cutoff (MWCO) value. In some embodiments, the MWCO may be in the range of about 1-100 kDa. In some embodiments, the MWCO may be in the range of about 10-100 kDa. In some embodiments, the MWCO may be in the range of about 20-80 kDa. In some embodiments, the MWCO may be in the range of about 30-50 kDa.

In some embodiments, the ultrafiltration/diafiltration unit utilized in this step may include at least one input and two outputs. One output may be used to collect the material not filtered through the membrane (in this case the immunoglobulins containing solution since the immunoglobulins (e.g., antibodies) are too large to pass through these membranes). Material may be passed repeatedly in this manner so as to facilitate a higher concentration and/or more effective buffer exchange. The second output collects the buffer solution that is filtered out through the membrane and is discarded into the interconnected waste vessel.

According to some embodiments, the resulting immunoglobulin fraction may be first concentrated to a required concentration. In some embodiments, the concentration may be between about 2-10% w/v immunoglobulins (such as, for example, IgG). In some embodiments, the concentration may be between about 3-8% w/v immunoglobulins. In some embodiments, the concentration may be between about 4-6% w/v immunoglobulins. In some embodiments, the concentration may be about 5% w/v immunoglobulins.

According to some embodiments, to facilitate such concentration, the immunoglobulins solution may be continuously/repeatedly passed through the ultrafiltration/diafiltration cassette or column (ultrafiltration/diafiltration unit). The solution is continuously filtered through the membrane, but only small molecules (such as, water and buffers, residuals of precipitation agents, etc.) can pass through the filter and into the filtrate stream, which is removed to the waste vessel, while the larger molecules (i.e., immunoglobulins (antibodies)) are retained in the solution. According to some embodiments, increasing or decreasing the filtration rate can be facilitated by controlling the fluid pressure/fluid flow in the system, for example, using one or more of the valves. For example, if the pressure is too low and there is very little filtration, closing or reducing the flow rate through the valve through which the retentate (the retained solution) is collected will increase the pressure on the filter and increase the flow through the filtrate valve, and vice versa. In some embodiments, in the system disclosed herein, the flow rate may be monitored by the lowering or increasing the flow rate using the peristaltic pump that is cycling the material through the filtration unit. In some embodiments, in-line pressure gauges may be utilized to further facilitate flow rate/pressure control. In some embodiments pressure may be manipulated to affect a lower or faster rate of filtration by the use of valves either on the input or any of the output lines of the ultrafiltration/diafiltration unit.

According to some embodiments, the solution buffer may be exchanged/replaced by a formulation buffer. To this aim, the formulation buffer may be added/inserted (for example, by the operation of a pump) at the same rate as the solution buffer that is being filtered. In such settings, using continuous/repeated diafiltration, greater than 99.5% of a permeable solute, such as buffer and residual precipitating agents (for example, caprylic acid, or sodium or chloride ions), may be removed by washing through one or more (for example, 4-8, for example, 6) volumes of the buffer exchanged solution.

According to some embodiments, a final desired concentration of purified immunoglobulins may be obtained by any of several alternative steps. For example, concentrating to a volume of a fixed percentage of the initial plasma volume that was introduced to the system. As the average plasma IgG concentration is relatively known in different populations, and while variable (e.g., between 8 -18 grams / liter), averaging of, for example, 10-12 plasma units can reduce the variability towards the population average. Such plasma pooling and averaging process can advantageously result in a consistent purification yield.

According to some embodiments, an in-line ultraviolet UV flow cell can be integrated into the kit or may be part of the device. Since purified antibodies have a known and characterized extinction coefficient for UV absorption (approximately 1.36-1.38 for a solution of 1 mg/ml at a wavelength of 280 nm), the use of such UV flow cell can enable accurately enable the system to arrive at an accurate final concentration of immunoglobulins.

According to some embodiments, a one-way valve may be utilized for sampling of the resulting solution, and the obtained sample may be used for measuring its extinction coefficient (in measuring unit that may be associated or be part of the device), for determining the resulting immunoglobulin concentration.

According to some embodiments, as detailed herein, the biological simple (e.g. plasma), is processed in a closed, sterile system. Accordingly, there may be no further need for additional filtration or sterilization. Nevertheless, if needed, the system can accommodate an additional, in-line sterilization filter, having a pore size of 0.22 micron or smaller, allowing further safety measures, to ensure sterility of the resulting purified, concentrated immunoglobulins composition.

According to some embodiments, once the process has completed, and the purified, concentrated, and optionally sterilized and/or filtered immunoglobulin composition has been collected in the collection vessel, the collection vessels may be disconnected/disengaged from the system, while maintaining its sterility. In some embodiments, for disconnecting/disengaging the collection bag, the tubing element connecting the bag to the kit, may be sealed (for example, by welding or clamping) at two locations/regions (i.e., proximal and distal), in order to enable the disconnection between the two locations and facilitate the sterile disconnection of the final purified immunoglobulin concentrate composition stored in the collection vessel. Once the collection vessel has been disengaged, it may be mobilized and used for any designated purpose in the same or a discrete location.

According to some embodiments, any suitable buffer may be used throughout the purification process. Such buffers include, for example, but not limited to: acetic acid, citric acid, NaCl, water (for example, sterile distilled water), TRIS, PBS, sodium acetate, HEPES, MOPS, Carbonate, Oxalate, glycine, proline, lysine, arginine, histidine, glutamate, succinate, citrate, lactic acid, adipic acid, MES, aspartic acid, and the like, or combinations thereof.

According to some embodiments, reagents used in the purification process may include such reagents as, but not limited to: caprylic acid, separation resins (e.g. anion or cation exchange resins, multi-mode resins, affinity resins), buffers, and the like.

In some embodiments, each buffer is stored/held/distributed from a discrete reagent vessel. In some embodiments, a mixture of two of more buffers may be stored/held/distributed from a discrete reagent vessel.

In some embodiments, the resulting purified, concentrated immunoglobulins solution/composition may include at least about 70% IgG, at least about 75% IgG, at least about 80% IgG, at least about 85% IgG, at least about 90% IgG, at least about 95% IgG, at least about 99% IgG.

According to some embodiments, the resulting purified, concentrated immunoglobulins solution/composition is an IVIG composition, i.e., IgG that is formulated to be delivered intravenously. According to some embodiments, the resulting purified, concentrated immunoglobulins solution/composition is an SCIG composition, i.e., IgG that is formulated to be delivered/administered subcutaneously. According to some embodiments, the resulting purified, concentrated immunoglobulins solution/composition is an IMIG composition, i.e., IgG that is formulated to be delivered/administered Intra-muscularly. In some embodiments, the resulting purified, concentrated immunoglobulins solution/composition of IgG may be formulated for suitable delivery route.

According to some embodiments, the resulting purified, concentrated immunoglobulins solution/composition is hyperimmune IgG (or HIG). In some embodiments, the HIG may be purified from any suitable plasma from immunized subjects such as, humans or animals.

HIG have several advantages as therapeutic agents, over the use of mere convalescent plasma (CP). HIGs are significantly more concentrated. The average IgG concentration in plasma is between 8 -18 grams/liter whereas a standard 5% HIG solution contains 50 grams/litter. Further, HIGs are less variable than individual units of CP since they are comprised of several donor units. HIGs contain a wider variety of antibodies due to the higher number of donors and would cover a larger number of epitopes in the target virus. Additionally, HIGs are safer for use due to several reasons: Transfusion-associated adverse events such as TRALI (Transfusion Associated Lung Injury) are associated with a high titer of anti-HLA or anti-HNA antibodies which are diluted in a pool comprised of several donors. Hemolytic reactions due to blood group ABO incompatibility are also mitigated by pooling due to both dilution of the isoagglutinin antibodies, and the neutralization of these by antibodies found in secretor phenotype blood units when several units of different blood type are mixed (especially when performing “smart pooling”).

In some embodiments, the purified immunoglobulin composition does not exhibit residual prothrombotic activity or said residual prothrombotic activity is below a detection threshold.

In some embodiments, the resulting purified immunoglobulins solution/composition may include less than about 50 mIU/ml of factor XIa (FXIa), less than about 30 mIU/ml, less than about 10 mIU/ml of FXIa, less than about 3 mIU/ml of FXIa.

According to some embodiments, the level of FXIIa (PKA) in the purified immunoglobulin fraction/composition/solution is less than about 100 IU/ml, less than about 50 IU/ml or less than about 35 IU/ml.

According to some embodiments, the level of plasminogen in the purified immunoglobulin fraction/composition/solution is less than about 0.01 gr/L.

According to some embodiments, the level of Alpha 1 antitrypsin in the purified immunoglobulin fraction/composition/solution is less than about 0.04 gr/L.

According to some embodiments, the level of albumin in the purified immunoglobulin fraction/composition/solution is less than about 0.002 gr/L.

According to some embodiments, the level of IgA in the purified immunoglobulin fraction/composition/solution is less than about 0.06 gr/L.

According to some embodiments, the level of IgM in the purified immunoglobulin fraction/composition/solution is less than about 0.2 gr/L.

According to some embodiments, the biological sample may include a plasma pool of at least 4 plasma units, at least 6 plasma units, at least 8 plasma units, at least 10 plasma units, at least 12 plasma units, at least 14 plasma units, at least 18 units, at least 24 units. In some embodiments, the plasma sample may include an amount of 4-12 plasma units. In some embodiments, the plasma is thawed prior to use.

In some embodiments, the plasma is convalescent plasma. In some embodiments, the plasma units may be obtained from one or more donors.

According to some embodiments, the systems and methods disclosed herein allow the purification of immunoglobulins from plasma sample in a time frame of about 6 to 48 hours. In some embodiments, the time frame may be in the range of about 8-36 hours. In some embodiments, the time frame may be in the range of about 12-24 hours. In some embodiments, the time frame may be in the range of about 8-12 hours. In some embodiments, the time frame is less than about 4 days.

According to some exemplary embodiments, a purification process configured to be executed by the system disclosed herein may include one or more of the following steps, in order to yield, highly purified, concentrated IgG, with minimal to no contaminants:
Thawing and mixing of plasma to obtain plasma pool;
Addition of a 1:1 volume/weight of buffer (such as, 100 mM acetic acid glacial);
Addition of an additional volume of precipitating agent (for example, 5% caprylic acid (CA)), and mixing for a time length, of, for example, 10-120 minutes. ;
Filtration through a crude filter (for example, about 1 cm² per 1 ml of input plasma) This step may be performed more than one time;
Washing of the filter cake with an additional 1 volume of plasma with buffer (for example, 40 mM acetic acid pH~4.5);
Loading on separation resin (for example, Cytiva CM Sepharose FF resin);
Washing the column (for example, with 40 mM acetic acid pH 4.5);
Eluting with an elution buffer (for example, 100 mM tris, 100 mM NaCl, pH 8.5) and collecting the eluate for the next step
Diluting with WFI at a 1:1 ratio
Performing a DEAE column separation:
Passing the diluted mixture through the DEAE column and collecting the unbound purified immunoglobulin solution
the final product after these steps is a highly purified (>99.9% monomer+dimer) IgG with no detectable impurities such as IgA (<0.06 gr/L).
Concentration and buffer exchanging to the desired formulation strength (final formulation buffer at pH 5-6, and a concentration of approximately 5% w/v of IgG).

Reference is now made to FIG. 5, which shows a block diagram of a method for purification of immunoglobulins utilizing the system as disclosed herein. As shown in FIG. 5, method 600 includes in step 602 providing blood plasma sample. As detailed herein, the blood plasma sample may be freshly obtained, or frozen and thawed plasma, and may include a pool of several plasma units, for a single or a plurality of users. In step 604, the system for purification is prepared, by associating a kit as disclosed herein with a device for purification of immunoglobulins, as disclosed herein. At step 606, the plasma sample vessel is fluidly connected to the purification system, which is consequently activated to execute a series of sequential purification steps, whereby, at step 608, fractions are sequentially and timely transferred between bio-process vessels, filtering units and/or separation columns of the system. At step 610, the purified concentrated immunoglobulins are collected in a dedicated collection vessel. At step 612, the collection vessel may be optionally sealed prior to being disengaged from the system, ready for further use.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

### EXAMPLES

### Example 1—Purification of Immunoglobulins From Plasma Using Caprylic Acid (CA) and Separation Columns(s)

In order to obtain IgG from plasma samples at a high purity, plasma (12 units, approximately 2.4 liters) was pooled and subjected to a purification process as per the steps below:
Addition of a 1:1 volume/weight of 100 mM acetic acid
Addition of an additional volume of 5% caprylic acid (CA) which is equivalent to 10% CA compared to the input plasma volume.
Mixing for 60 minutes.
Filtration through a crude Whatman filter with an area of approximately 1750 cm²
Washing of the filter cake with an additional 1 volume of plasma (approximately 2.4 liters) with 40 mM acetic acid pH~4.5
Loading the combined filtrate on a binding mode separation resin (300-600 ml of CM Sepharose Fast Flow (Cytiva)) in order to bind the immunoglobulins.
Washing the column with 40 mM acetic acid pH 4.5
Eluting with 100 mM tris, 100 mM NaCl, pH 8.5 and collection of the elution fraction (the immunoglobulins bind to CM cation exchange resin and require elution using a high salt and high pH buffer).
Diluting with WFI at a 1:1-1:2 ratio
Performing a DEAE column separation. The DEAE binds mostly contaminating proteins and very little of the desired immunoglobulins. Therefore, the unbound fraction (flow-through and wash combined) is collected. Variable amounts of the product were loaded on variable amounts of DEAE Sepharose Fast Flow (Cytiva) or for final analysis. The actual amounts of material and of DEAE resin were lower to enable performing several experiments from the same input material. For example, ¼ of the volume was loaded on 30 ml of DEAE resin which is equivalent to loading the entire material on 120 ml of DEAE resin.

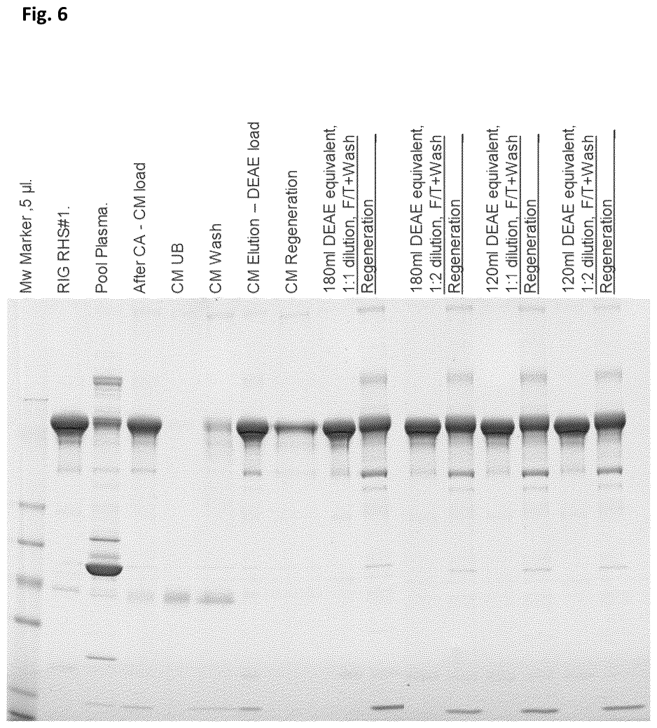

The results are presented in FIG. 6, which shows an SDS-PAGE analysis of the purity of the product. The different steps of the exemplary process are labeled:
Mw marker is a protein standard with known molecular weights for reference (the molecular weight of an IgG molecule is approximately 150 kDa);
RIG RHS #1 is control of a purified immunoglobulin commercial product (KedRab/KamRab anti-rabies);

### Pool Plasma;

After CA—CM load is the pool plasma after treatment with caprylic acid and subsequent filtration, greatly reducing the levels of contaminating proteins (especially albumin) and which serves as the load fraction for the cation exchange purification step using the CM cation exchange resin;
CM UB is the fraction of the purification process that is not bound (unbound=UB) to the CM resin and is mostly contaminant proteins and not IgG;
CM wash is the collected wash buffer used to remove additional unbound proteins from the CM column to which the IgG proteins are bound;
CM elution-DEAE load is the cation-exchange chromatography purified IgG elution fraction that is also used (after additional dilution) as the input fraction for the next purification step of the DEAE anion exchange column;
CM regeneration is the residual IgG that are found at low amounts on the CM resin column and were not successfully eluted from the column using the relatively mild buffer used in the process but are removed via high salt (1M NaCl) regeneration and subjected to analysis;
The next four sets of protein lanes are labeled as [X]ml DEAE equivalent, [Y:Z] dilution F/T+wash, and then the respective regeneration steps. [X] indicates the DEAE equivalent volume used for this step after calculating the initial volume taken for the purification experiment from the entire previous step. For example—if ¼ of the total volume of the previous step was loaded on a 45 ml DEAE anion exchange resin column, the [X] value would be 45/¼ which is equivalent to 180 ml. [Y:Z] indicate the ratio of the dilution of the previous step output (CM elution) to water [1:1 or 1:2]. The label ‘F/T+Wash’ is added to indicate that the flow-through (unbound) fraction and a respective additional wash (with a similar buffer to the elution buffer after a 1:1 dilution with water:
approximately 50 mM Tris and 50 mM NaCl, pH~8.2) are collected from the DEAE column which binds the impurities (as well as aggregates and fragments) but allows IgG molecules to flow through the column. Regeneration of the column is performed and collected as for the CM column before, with 1M NaCl (high salt) to display the proteins bound by the DEAE column and removed from the IgG solution. Some IgG is also bound to the DEAE column as seen in these lanes, as are additional non-characterized protein bands.

As can be seen in FIG. 6, after loading on DEAE at amounts equivalent to 120-180 ml of DEAE resin, the purified fraction (the unbound immunoglobulins-F/T+wash) displays a single band. A high salt (1M NaCl) regeneration of the column is able to remove undesired additional protein bands that are adhered to the DEAE resin and removed from the immunoglobulin extract. The column can be reused afterwards as the 1M NaCl was able to remove all residual proteins from the column (not shown), e.g. in such cases where a small volume column would be reused several times in the same purification.

In addition, prothrombotic activity in the fraction of PKA (FXIIa) was found to be very low, below the limit of detection for FXIa (<0.6 mIU/mL).

The IgG solution composition was next concentrated using a centrifugal 30 kDa Molecular Weight Cutoff Centricon device. The concentration of the IgG solution to approximately 4%-6% (40-60 mg/mL) was performed in order to analyze potential residual contamination by IgA. Very little IgA was measured (<0.2 gr/L).

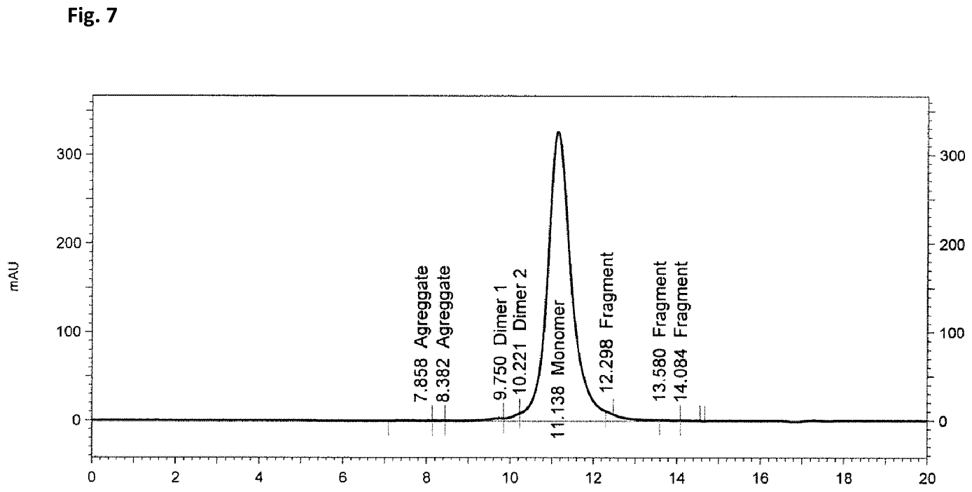

The final product was highly purified IgG: >98% monomer+dimer as judged by molecular size distribution (MSD), presented in FIG. 7.

In repeat experiment, double amounts of DEAE equivalent resin were used (½ of the material was loaded on 200 ml of the DEAE resin, or 400 ml DEAE resin equivalent for the entire material) which reduced IgA levels in the final product to below detection threshold (<0.06 gr/L). Also, IgG purity was even higher: >99.9% monomer+dimer, with <0.1% combined aggregates and fragments.

The MSD (Molecular Size Distribution) analysis by HPLC of the final product after utilizing 400 ml of DEAE resin equivalents, exhibited >99.9% monomers+dimers, with <0.01% aggregates and fragments combined, as can be seen in FIG. 7.

IgG subunit distribution was measured compared to other IgG pharmaceutical products. The distribution of IgG 1-4 was: IgG1—67-72.3%, IgG2—22.8-28.7%, IgG3—1.8%-2.1%, IgG4—2.2-3.0%. A commercial IVIG product (Cytogam®) tested for comparison and found to have a similar distribution: IgG1—71.6-73.5%, IgG2—20.8-23.3%, IgG3—3.3-4.8%, and IgG 4—1.7-2.1% (results for three different batches).

This indicates there is no major difference between the final product obtained using the method described herein for the purification system, and a product that is manufactured using classic fractionation technology in a GMP facility. By simple application of higher amounts of DEAE resin equivalents an extremely high purity can be achieved. This can be performed by either utilizing columns of approximately 400 ml volume in the device for a batch minipool size of 12 plasma units, or using smaller columns and reusing these columns, regenerating them with (e.g.) 1M NaCl in between loading cycles.

### Example 2—Sterilization of Purified Immunoglobulins From Plasma

The final IgG DEAE eluate generated as detailed in Example 1, was subjected to a pasteurization. To this aim, a constant amount of stabilizer (such as sorbitol 15%) and heating to a controlled temperature (50° C. or 60° C.), can reduce dangerous pathogens as well as qualify as an additional 2nd viral inactivation method (the 1st being the caprylic acid purification). To this aim, the purified IgG composition was incubated with 15% sorbitol (w/v) for 1-5 hrs. at temperatures of 50° C. and 60° C. It was tested whether the pasteurization can be performed in a Tris buffer at pH~8.2 (direct addition of the sorbitol into the DEAE output) or if it may be improved by changing the buffer to a formulation buffer (40 mM acetate pH 6.0, 140 mM NaCl with 15% sorbitol). In order to assess the effect of pasteurization on product stability both the anti-Covid titer (using an ELISA based method), and the level of aggregates formed using MSD-HPLC were tested.

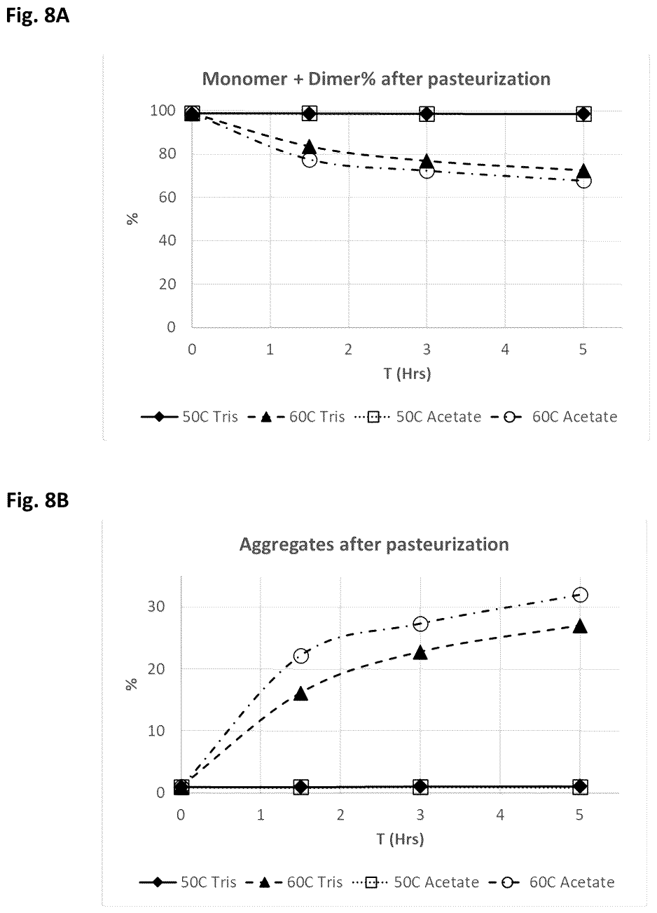

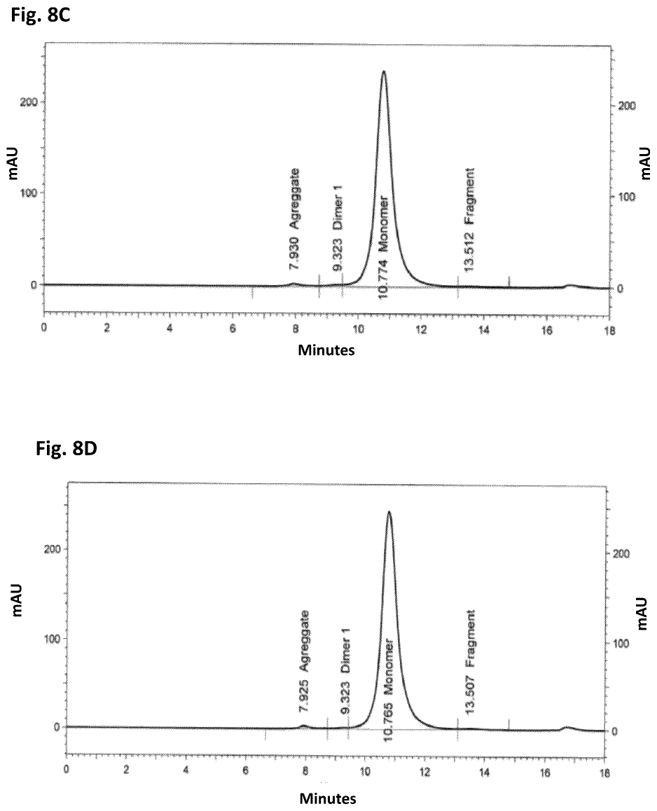

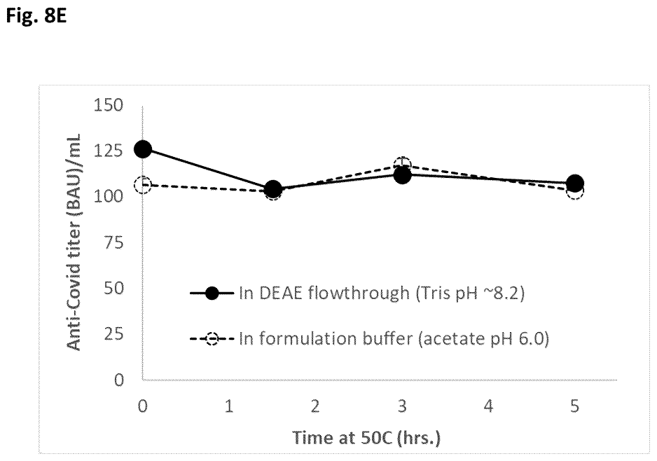

The results are presented in FIGS. 8A-E. The results show that after the DEAE step there are essentially very few aggregates (1%), and almost all of the IgG is monomeric and dimeric (active forms; 98%). Incubation up to 5 hrs. at 50° C. with 15% sorbitol did not cause any detectable damage to the protein, but at 60° C. aggregates quickly formed. Two buffers were tested, namely, acetate and Tris, with similar results. This indicates that buffer exchange can be performed after all steps including 50° C. pasteurization have been finalized. Anti-Covid activity test confirmed these results. Activity remained similar at 50° C. regardless of the buffer system, over the entire 5 hours duration of pasteurization.

1.-39. (canceled)

40. A device for automatic purification and concentration of immunoglobulins from a blood plasma sample, the device comprising:
one or more pumps;
one or more valves; and
a plurality of connecting elements configured to hold corresponding buffer vessels and/or bio-process vessels,
wherein, when said plasma sample is introduced to the device, a sequential purification process is facilitated, whereby plasma sample fractions are transferred between one or more of the bio-process vessels, such that immunoglobulins are purified, concentrated and collected at a collecting vessel, wherein the transferring of the fractions is facilitated by a coordinated operation of the one or more pumps and the one or more valves.

41. The device according to claim 40, wherein the device is portable.

42. The device according to claim 40, further comprising a control unit configured to control operation of the one or more pumps and one or more valves.

43. The device according to claim 40, wherein the purified immunoglobulins comprise hyper immune IgG (HIG).

44. The device according to claim 40, wherein the purified immunoglobulins comprise at least about 90% IgG.

45. The device according to claim 40, wherein the purified immunoglobulin do not exhibit residual prothrombotic activity and/or wherein the level of FXIa is less than about 10 mlU/ml and/or wherein the level of FXIIa (PKA) in the purified immunoglobulin fraction is less than about 35 IU/ml.

46. The device according to claim 40, wherein the level of one or more of: plasminogen, Alpha 1 antitrypsin, albumin, IgM and IgA in the collected immunoglobulin fraction is below a respective detection level, wherein the detection levels comprise: less than 0.01 gr/L for plasminogen, less than <0.04 gr/L for Alpha 1 antitrypsin, less than 0.0022 gr/L for albumin, <less than 0.05 gr/L for IgM, and/or less than 0.06 gr/L for IgA.

47. The device according to claim 40, wherein the device is configured to allow purification of immunoglobulins in a time frame of about 6-48 hours.

48. The device according to claim 40, wherein one or more of the elements comprise weight transducers configured to determine the weight of a connected vessel.

49. A sterile kit for automatic purification and concentration of immunoglobulins from blood plasma sample, the kit comprising:
a plurality of reagent vessels, comprising one or more mixtures for use during the purification process;
a plurality of bio-process vessels, configured to facilitate purification steps;
one or more filtration units; and
one or more separation columns;
wherein the kit is configured to associate with a corresponding separation device, to allow sequential transfer of plasma sample fractions between the bio-process vessels, one or more filtration units and/or the one or more separation columns, to result in purified concentration immunoglobulin composition, collected at a collection vessel.

50. The kit according to claim 49, further comprising tubing elements configured to allow fluid connection between reagent vessels, bio-process vessels, filtration unit(s) and/or separation column(s).

51. The kit according to claim 49, wherein the bio-process vessels are configured to allow/accommodate incubation, mixing and/or reaction steps of the purification process.

52. The kit according to claim 49, wherein the vessels are made of nylon, plastic, PVC, silicon, PP, PES, BPC, HDPE, or any combination thereof.

53. The kit according to claim 49, wherein the vessels comprise one or more connections, allowing connecting between the vessels and/or between components of the separation device, using tubing elements.

54. The kit according to claim 49, wherein the separation columns comprise ion exchange columns.

55. The kit according to claim 54, wherein the ion exchange columns comprise a resin having a functional group selected from: Carboxymethyl cellulose (CM), Sulfonate(S), Sulfopropyl (SP), diethylaminoethyl (DEAE), quaternary aminoethyl (QAE), diethylaminopropyl (ANX), and/or quaternary ammonium (Q).

56. The kit according to claim 49, further comprising a collection vessel, configured to collect/hold the purified immunoglobulins.

57. The kit according to claim 49, wherein the immunoglobulin composition comprises IgG having a purity of over about 90%.

58. A system for purification of immunoglobulins from a blood plasma sample, the system comprising the device according to claim 40, and a sterile kit comprising:
a plurality of reagent vessels, comprising one or more mixtures for use during the purification process;
a plurality of bio-process vessels, configured to facilitate purification steps;
one or more filtration units; and
one or more separation columns.

59. A method for automatic purification of IgG from a blood plasma sample, the method comprising:
providing a plasma sample in a vessel to the system according to claim 58;
allowing fractions of the plasma sample to transfer sequentially between bioprocess vessels, one or more filtration units and/or one or more separation columns; and
collecting the purified IgG, in a collection vessel.
